(19) United States
(12) Patent Application Publication
(10) Pub. No.: US 2019/0352259 A1
(43) Pub. Date: Nov. 21, 2019

(54) LACTAM COMPOSITIONS

(71) Applicant: Conopco, Inc., d/b/a Unilever, Englewood Cliffs, NJ (US)

(72) Inventors: Panagiotis KOTSAKIS, Manchester (GB); Neil James PARRY, Tarporley (GB)

(21) Appl. No.: 16/461,636

(22) PCT Filed: Oct. 19, 2017
(86) PCT No.: PCT/EP2017/076718

§ 371 Date: May 16, 2019

(30) Foreign Application Priority Data
Nov. 17, 2016 (EP) 16199262.3

(51) Int. Cl. C07D 207/38; A01N 43/36; A61K 8/49; A61Q 17/00

(52) U.S. Cl. CPC C07D 207/38; A61Q 17/005; A61K 8/4913; A01N 43/36

(57) ABSTRACT

Compositions comprising a lactam. Pharmaceutical compositions comprising such lactams for use in methods of treatment.

The present invention relates to compositions comprising lactams. The compositions are suitable for use as anti-microbial, anti-biofilm and bacteriostatic compositions, and particularly for use as personal care compositions.

WO 2007/085042 and WO 2004/016588 disclose lactams for antimicrobial benefit and steps towards their synthesis. WO2014/118240 discloses antimicrobial compositions comprising a lactam and a hydrotope.

The present invention relates to novel lactams having anti-microbial activity.

These lactams may exhibit desirable anti-bacterial and/or bacteriostatic activity. The compounds may be especially useful as the inventors believe that the lactams of the invention may be used for population control of bacterial colonies without triggering the mechanisms that are thought to lead to evolutionary resistance.

Accordingly, in a first aspect, the present invention may provide a composition comprising a lactam, wherein the lactam is a lactam selected from:

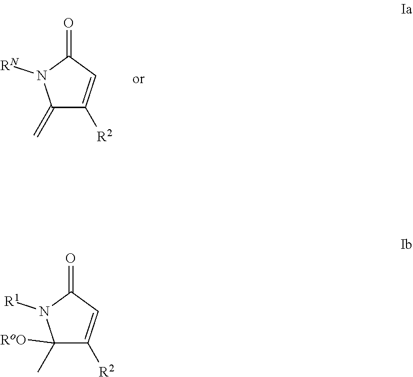

wherein
R¹ is selected from H, R^{a} and R^{N};
each R^{O} and R^{N} is —X—(CH₂)ₙ—R^{T};
X is a bond or C═O;
n is an integer from 1 to 8;
R^{T} is OR^{a}, NR^{a}₂, NR^{a}₃⁺, NR^{a}(CO)CR^{a}═CR^{a}₂;
each R^{a} is independently H or C₁₋₄alkyl; and
R² is optionally substituted phenyl.
   NR^{a}₃⁺ groups have a counter ion, for example an iodide (I⁻).

In some embodiments, the lactam is a lactam of formula Ia. In some embodiments, the lactam is a lactam of formula IIa.

Where used herein, optionally substituted may refer to substituents selected from the following: R^{a}, halogen, OR^{a}, NR^{a}₂, COOR^{a}, NR^{a}COCR^{a}═CR^{a}₂, CONR^{a}₂, where each R^{a} is independently H or C₁₋₄alkyl, preferably H or methyl. Preferred optional substituents include F, Cl, Br, OH, OMe, COOMe, and NHCOCH═CH₂.

R² is a phenyl group or a substituted phenyl group, for example, a mono-substituted phenyl group. Substitution may be ortho, meta, or para. Preferably, it is para. Preferred substituents include halogen and methyl. For example, and without limitation, R² may be selected from phenyl, 4-fluorophenyl, 4-chlorophenyl, 4-bromophenyl and 4-methylphenyl. In particularly preferred embodiments, R² is 4-chlorophenyl.

In some embodiments, R^{T} is selected from —OH, —N⁺Me₃, and —NH(CO)CH═CH₂.

In some embodiments, the lactam is a lactam of formula Ib and R¹ is H or R^{a}; preferably H or methyl; most preferably H. In other words, preferably, there is only one —X—(CH₂)ₙ—R^{T} group in the lactam.

In some embodiments, the lactam is a lactam of formula Ib and X is a bond. In other words, R^{O} is preferably —(CH₂)ₙ—R^{T}. In some embodiments, R^{O} is —(CH₂)₂NMe₃⁺.

n is selected from 1, 2, 3, 4, 5, 6, 7, and 8. In some embodiments, n is 2. In some embodiments, n is 6.

For example, R^{N} may be —(CH₂)₂NMe₃⁺, —(CH₂)₆OH, or —(CH₂)₂NH(CO)CH═CH₂. In each of these X is a bond.

For example, R^{N} may be —CO—(CH₂)₂NH(CO)CH═CH₂. In this moiety, X is —(CO)—.

The composition may be aqueous or non-aqueous. The composition may be an emulsion.

The composition may be, without limitation, any of a personal care composition, a homecare composition, a pharmaceutical composition, or an industrial composition such as an anti-biofilm coating or paint, for example, for use in maritime environments. The composition may also be an agricultural chemical. The compositions may be suitable for use as antimicrobial, anti-biofilm and bacteriostatic compositions. Non-limiting examples of such compositions are provided herein. The compositions may also be used as additive compositions; in other words, the composition may be combined with further ingredients such as excipients to form a composition as described above.

It will be appreciated that the compositions are suitably personal or homecare compositions. In some cases, the composition is a personal care composition intended for use on the skin, for example, a skin cream, a cleanser, or a serum. In some cases, the composition is a homecare composition to be used in the home, for example a laundry liquid or cleaning product.

Accordingly, in some cases the composition further comprises a perfume ingredient, for example an encapsulated perfume. The amount of fragrance may be 0.01-1.5% wt. of the composition. Where a fragrance is encapsulated, the amount may be lower, for example, 0.01-0.5% wt.

In some cases, the composition is a pharmaceutical composition.

The invention therefore further provides use of a composition as described herein for use in a method of treatment of a human or animal subject. Method of treatment, as used herein, includes prophylaxis.

The method of treatment may be treatment of an infection caused by a gram-negative bacteria. The method of treatment may be treatment of a bacterial infection of a skin lesion in a subject. The method of treatment may be treatment of respiratory tract infections.

Preferably the composition contains 0.000001 to 50% wt. lactam, more preferably 0.001 to 50% wt. even more preferably 0.01 to 5% wt., most preferably 0.01 to 2%.

Exemplary Compounds Include:

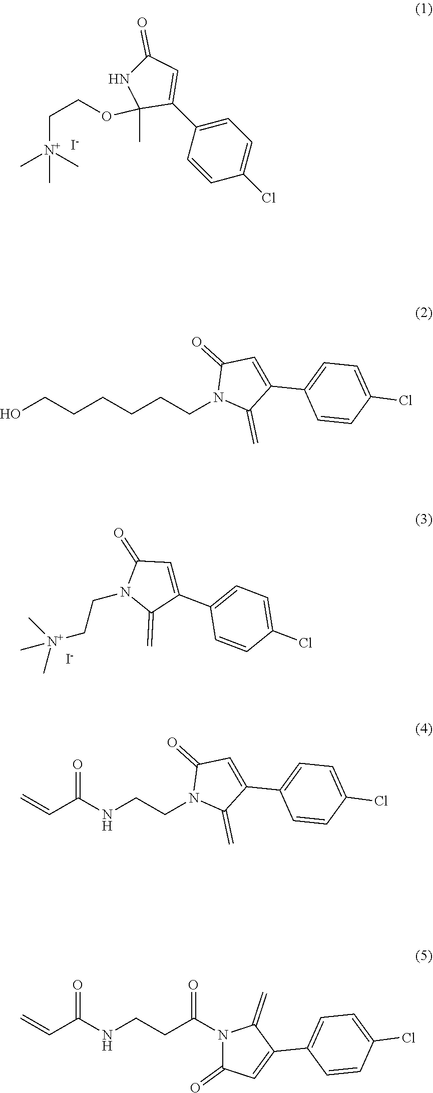

### DESCRIPTION

Compositions

The compositions described herein may be compositions having anti-microbial activity. In some cases, the compositions are anti-bacterial. They may have bactericidal and/or bacteriostatic activity. The inventor(s) have observed desirable bacteriostatic activity. Accordingly, in some cases, the composition is a bacteriostatic composition.

The compositions may also prevent and/or inhibit biofilm formation. Biofilms are formed when microorganisms stick to a surface. Biofilm extracellular polymeric substances may be formed. Biofilms (also referred to as slime) present problems in industrial environments; for example, they may form in pipes in apparatus, or industrial and agricultural structures, and on boat hulls and other marine structures. Biofilms may also pose a problem in domestic environments. For example, biofilms may form in domestic appliances such as washing machines. Biofilms are also present in personal care, for example, they may form on tooth surfaces.

Compositions suitable for any and all of these applications are within the scope of the invention. In some cases, the composition is a paint or other coating. In such cases, the composition may further comprise a binder, optionally a pigment and optionally one or more conventional additives (for example, to modify surface tension, improve flow properties, improve the finished appearance, increase wet edge, improve pigment stability, etc—such additives are known in the art). The composition may comprise an aqueous solvent or an organic solvent to suit purpose.

The composition may also be used in medical applications, for example to coat equipment.

In some cases, the composition is a pharmaceutical composition. In other words, the composition may comprise a lactam as described herein and a pharmaceutically acceptable excipient. The composition may be suitable for topical use (for example, it may be a cream or lotion), it may be suitable for ocular use (for example, it may be an used as a pharmaceutical eye drop), it may be suitable for optic use (for example, it may be used as an ear drop), it may be suitable as a mouth wash, or it may be suitable for oral or parenteral administration. In some cases, it may be suitable for insufflation/inhalation.

In some cases, the composition is a composition suitable for use in the home (often referred to as a homecare composition). Homecare compositions include, without limitation, cleaning products, laundry detergents, and fabric conditioners. In some cases, the composition is a homecare composition, for example a laundry liquid. The composition may therefore comprise a detergent surfactant and a builder. The composition may be a fabric conditioner (also called a fabric softener) and may comprise an antistatic agent. The composition may also be a domestic cleaning product.

In some cases, the composition is a personal care composition. For example, the composition may be intended for use on the skin (for example, a cream, cleanser or serum). For example, the composition may be useful in the prevention or treatment of acne. For example, the composition may comprise one or more of dimethicone, petrolatum, a humectant such ashyaluronic acid or glycerin; and ceramide(s). In some cases, the composition is a personal care composition comprising a detergent, for example, the composition may be a face wash or shower gel.

In some cases, the composition is a contact lens cleaning fluid.

The composition may be a composition suitable for use in agriculture, for example, as a soil additive (solid or liquid).

Methods of Treatment

The lactams described herein may be useful in methods of treatment of infections caused by gram negative bacteria.

The lactams described herein may be useful in methods of treatment (including prophylaxis) of bacterial infections of a skin lesions in a subject. The infection may be caused by gram negative or gram positive bacteria, or both.

Gram-Negative Bacteria

The proteobacteria are a major group of gram-negative bacteria, including Escherichia coli (E. coli), Salmonella, Shigella, and other Enterobacteriaceae, Pseudomonas, Moraxella, Helicobacter, Stenotrophomonas, Bdellovibrio, acetic acid bacteria, Legionella etc. Other notable groups of gram-negative bacteria include the cyanobacteria, spirochaetes, green sulfur, and green non-sulfur bacteria.

Medically relevant gram-negative cocci include the four organisms that cause a sexually transmitted disease (Neisseria gonorrhoeae), a meningitis (Neisseria meningitidis), and respiratory symptoms (Moraxella catarrhalis, Haemophilus influenzae).

Medically relevant gram-negative bacilli include a multitude of species. Some of them cause primarily respiratory problems (Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella typhi).

Gram-negative bacteria associated with hospital-acquired infections include Acinetobacter baumannii, which cause bacteremia, secondary meningitis, and ventilator-associated pneumonia in hospital intensive-care units.

Accordingly, the gram-negative bacteria may be selected from Escherichia coli (E. coli), Salmonella, Shigella, and other Enterobacteriaceae, Pseudomonas, Moraxella, Helicobacter, Stenotrophomonas, Bdellovibrio, acetic acid bacteria, Legionella, cyanobacteria, spirochaetes, green sulfur, and green non-sulfur bacteria, Neisseria gonorrhoeae, Neisseria meningitidis Moraxella catarrhalis, Haemophilus influenzae, Klebsiella pneumoniae, Legionella pneumophila, Pseudomonas aeruginosa, Escherichia coil, Proteus mirabilis, Enterobacter cloacae, Serratia marcescens, Helicobacter pylori, Salmonella enteritidis, Salmonella typhi, Acinetobacter baumannii.

Pseudomonas aeruginosa

Preferably, the gram-negative bacteria is a P. aeruginosa.

There are a number of P. aeruginosa strains, including PA01, PA7, USBPP-PA14 and strain 2192. Except where indicated otherwise, a reference to P. aeruginosa is intended to refer to any and all strains.

The methods described herein may be directed to treatment of infections in which P. aeruginosa is implicated. The P. aeruginosa may be a strain that produces AQs (alkylquinoline compounds). The P. aeruginosa may be a strain that produces one or both of PQS (Pseudomonas quinolone signal; 2-heptyl-3-hydroxy-4-(1H)-quinolone) and HHQ (4-hydroxy-2-heptylquinoline). The P. aeruginosa may be a strain belonging to one of the two major P. aeruginosa genomic groups (PAO1 and PA14).

The lactams described herein may be useful in methods of treatment of infections caused by gram negative bacteria.

Skin Lesions

As described herein, in one aspect the invention relates to methods of treatment for skin lesion infections. These are commonly referred to as non-healing wounds. A chronic condition is characterised by long duration or frequent recurrence, typically where a wound has not healed within 12 weeks of first occurrence.

Chronic bacterial wound infections present a particular problem in diabetes sufferers. Accordingly, the present invention is envisaged for the treatment of bacterial skin lesion infections in patients that have been diagnosed with diabetes.

In some cases, the bacterial infection is an infection in which Pseudomonas, (usually but not necessarily P. aeruginosa) is implicated.

The skin lesion may be a chronic skin lesion.

The subject may have diabetes.

As used herein, the term skin lesion refers to a wound or sore resulting from, for example, an injury, other skin trauma or a pathological cause such as restricted blood circulation.

Examples of skin lesions include areas where the skin has been torn, cut or punctured, ulcers (from pre-ulcerative lesions to gangrenous tissue).

Skin lesions may affect only the epidermis and dermis, or they may affect tissues all the way to the fascia.

Respiratory Tract Infections

The lactams described herein may be useful for the treatment of a variety of respiratory tract infections in a subject, but may be especially useful for the treatment of bacterial lower respiratory tract infections such as lung infections. The bacterial infection may result in an acute or chronic lung infection, or may cause a recurring lung infection and thus treatment is of an acute, chronic and/or recurring lung infection.

The methods described herein may be especially suitable for patients having cystic fibrosis. Accordingly, the method may be a method of treating a subject who has been diagnosed with cystic fibrosis. Cystic fibrosis is a genetic disorder inherited in an autosomal recessive manner. Sufferers have mutations in both copies of the CTFR gene (for the cystic fibrosis transmembrane conductance regulator (CFTR) protein) meaning that CFTR is not functional. The most common mutation is AF508.

As used herein, the terms cystic fibrosis sufferer and cystic fibrosis patient refer to a subject who has been medically diagnosed with cystic fibrosis and I or has mutations in copies of the CTFR gene.

In some subjects, and particularly in subjects diagnosed as having cystic fibrosis, the lactams may be used prophylactically for preventing the occurrence or recurrence of a respiratory tract infection.

Biofilm

The term “biofilm” as used herein refers to biological films that develop and persist at interfaces in aqueous environments. These biological films are composed of microorganisms embedded in organic gelatinous matrices composed of one or more matrix polymers that are secreted by the resident microorganisms. Biofilms can develop into macroscopic structures and are also capable of trapping nutrients and particulates that can contribute to their enhanced development and stability. Biofilms can also prevent penetration of antimicrobial agents, which may lead to persistent infections. Formation of biofilms provides bacteria with a protected environment can withstand various stresses, including many antibiotic treatments.

Biofilm formation enables the bacteria to resist antibiotics because once the bacteria sense that the outer layer of the biofilm is being destroyed, the inner layers will grow stronger to re-establish the community. The present invention is based on the inventors' understanding of the properties of certain lactams as described herein and their insight into the way in which said lactams may influence quorum sensing in bacteria such as P. aeruginosa.

Methods of Treatment

It will be appreciated that the term “methods of treatment” as used herein includes prophylaxis, treatments that hamper bacterial colony population growth, treatments that keep a bacterial colony population stable, and treatments that reduce or eradicate a bacterial population.

The lactams of the invention may be useful in the long term treatment of infections.

In some cases, the bacterial infection is an infection in which Pseudomonas, (usually but not necessarily P. aeruginosa) is implicated.

The methods described herein may be suitable for long term use. Accordingly, the methods may include regular administration of the lactam to a subject for a period of at least several weeks, several months, at least one year, at least two years, at least three years, at least 5 years, at least 8 years, or at least 10 years.

It will be appreciated that, except where expressly provided otherwise, all preferences are combinable.

1. A composition comprising a lactam selected from: 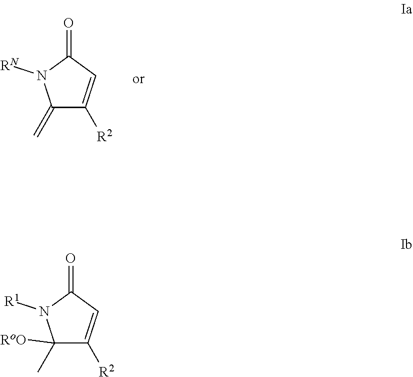
wherein
R¹ is selected from H, R^{a} and R^{N};
each R^{O} and R^{N} is —X—(CH₂)ₙ—R^{T};
X is a bond or C═O;
n is an integer from 1 to 8;
R^{T} is OR^{a}, NR^{a}₂, NR^{a}₃⁺, NR^{a}(CO)CR^{a}═CR^{a}₂;
each R^{a} is independently H or C₁₋₄alkyl; and
R² is optionally substituted phenyl.

2. The composition of claim 1, wherein R² is mono-substituted phenyl.

3. The composition of claim 1, wherein R^{T} is selected from —OH, —N⁺Me₃, and —NH(CO)CH═CH₂.

4. The composition of claim 1, wherein the lactam is a lactam of formula Ib and wherein R¹ is H or R^{a} and/or X is a bond.

5. The composition of claim 1, wherein n is 2.

6. The composition of claim 1, wherein the lactam is selected from:

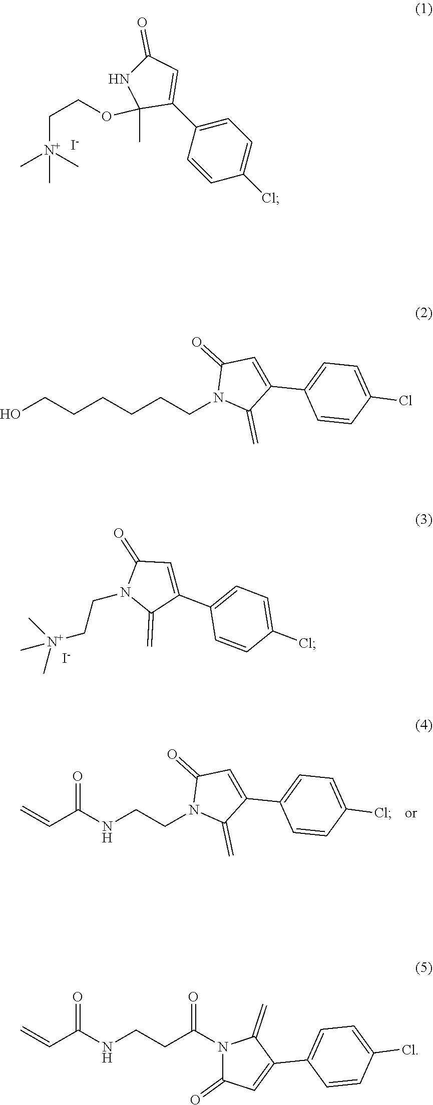

7. The composition of claim 1, wherein the composition is aqueous.

8. The composition of claim 1, wherein the composition is an emulsion.

9. The composition of claim 1, wherein the composition is a personal care composition, optionally wherein the composition is a skin cream, a cleanser, or a serum.

10. The composition of claim 1, wherein the composition is a homecare composition, optionally wherein the composition is a laundry liquid or a cleaning product.

11. The composition of claim 1, wherein the composition comprises a perfume.

12. The composition of claim 1, wherein the composition is a pharmaceutical composition.

13. (canceled)

14. (canceled)

15. (canceled)

16. The composition of claim 1, wherein R² is 4-chlorophenyl.

17. A method of treatment of an infection caused by a gram-negative bacteria in a subject, comprising administering to the subject the composition of claim 1.

18. A method of treatment of a bacterial infection of a skin lesion in a subject, comprising administering to the subject the composition of claim 1.

19. A method of treatment of a respiratory tract infection in a subject, comprising administering to the subject the composition of claim 1.

20. The composition of claim 11, wherein the perfume is encapsulated.
